(19) United States
(12) Patent Application Publication
(10) Pub. No.: US 2024/0194886 A1
(43) Pub. Date: Jun. 13, 2024

(54) CONDUCTIVE MATERIAL DISPERSION FOR SECONDARY BATTERY AND SLURRY COMPOSITION FOR SECONDARY BATTERY ELECTRODE CONTAINING THE SAME

(71) Applicant: CARBON T&C Co. Ltd., Seoul (KR)

(72) Inventors: Gun Soo KIM, Seoul (KR); Ho Kyoung CHOI, Seoul (KR); Young Bin CHOI, Seoul (KR)

(73) Assignee: CARBON T&C Co. Ltd., Seoul (KR)

(21) Appl. No.: 18/513,967

(22) Filed: Nov. 20, 2023

(30) Foreign Application Priority Data
Nov. 28, 2022 (KR) 10-2022-0161509

(51) Int. Cl. H01M 4/62; C07D 207/267; H01M 4/02; H01M 4/48; H01M 4/583

(52) U.S. Cl. CPC H01M 4/625; C07D 207/267; H01M 4/48; H01M 4/583; H01M 2004/027

(57) ABSTRACT

The present invention relates to a conductive material dispersion for a secondary battery, and a slurry composition for the electrodes of a secondary battery comprising same. Specifically, the conductive material dispersion comprising a vinyl-based dispersant to show better performance than existing dispersants can be used for the electrodes of a secondary battery.

### TECHNICAL FIELD

The present invention relates to a conductive material dispersion for a secondary battery, and a slurry composition for the electrodes of a secondary battery comprising same. Specifically, the conductive material dispersion comprising a vinyl-based dispersant to show better performance than existing dispersants can be used for the electrodes of a secondary battery.

### BACKGROUND ART

Japanese Patent Application Publication No. JP 2021-072279 A is an invention relating to dispersing a conductive material in a dispersing medium, but it is not optimized for dispersing a conductive material. In addition, European Patent Application Publication No. EP 3 770 204 A1 defines a dispersant as a polymer whose structure contains an aromatic vinyl monomer unit and a straight-chain alkylene structural unit having 4 or more carbon atoms. However, when the dispersant has such structure, there is a problem that it is inadequate for dispersing a conductive material whose surface has hydroxyl groups.

### PRIOR ART CITATION

Japanese Patent Application Publication No. JP 2021-072279 A

European Patent Application Publication No. EP 3 770 204 A1

### SUMMARY OF INVENTION

### Technical Problem

The present invention is to define the structure of a dispersant that facilitates dispersing conductive materials such as carbon nanotubes, graphene, and carbon black in a medium and to provide a conductive material dispersion that induces uniform dispersion in the negative electrode slurry of a secondary battery and improves electrical connection to improve the lifespan and efficiency of secondary batteries. In addition, a conductive material pre-dispersion slurry for the electrodes of a secondary battery with improved dispersion characteristics of the conductive material can be implemented. In particular, a conductive material pre-dispersion slurry for the electrodes of a secondary battery that has a relatively high content of the conductive material, has a relatively low viscosity, and can be easily manufactured through a relatively simple process can be implemented. By applying the conductive material pre-dispersion slurry, an electrode with excellent performance can be manufactured, and a secondary battery using the electrode can be manufactured.

### Solution to Problem

The dispersant according to an embodiment of the present invention may comprise the following formula 1:

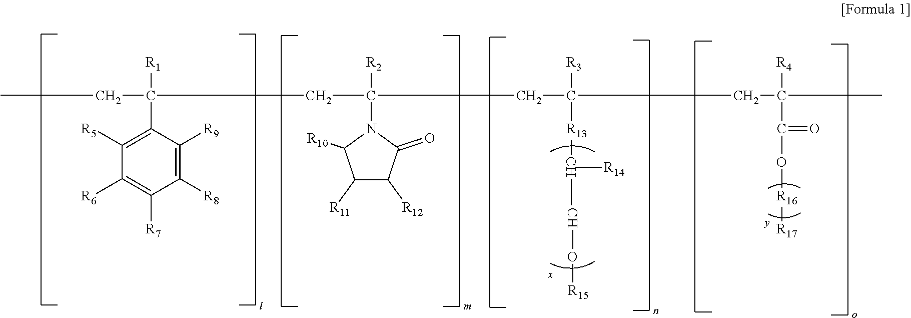

wherein R₁ to R₄ may each independently be hydrogen or methane,
R₅ to R₁₂ independently comprise at least one aliphatic or aromatic hydrocarbon group or halogen element that comprises or not comprises linear, branched, or cyclic heteroatoms having 1 to 20 carbon atoms that comprise or not comprise hydrogen or unsaturated groups,
R₁₃ comprises at least one alkyl or ester group having 1 to 3 carbon atoms comprising a heterogroup,
R₁₄ is independently hydrogen or methyl,
R₁₅ is an aliphatic or aromatic hydrocarbon group that comprises or not comprises linear, branched, or cyclic heteroatoms having 1 to 20 carbon atoms that comprise or not comprise hydrogen or unsaturated groups,
R₁₆ is an aliphatic or aromatic hydrocarbon group that comprises or not comprises linear, branched, or cyclic heteroatoms having 1 to 20 carbon atoms that comprise or not comprise unsaturated groups,
R₁₇ is one selected from the group consisting of hydrogen and carboxylic acid.
x is an integer from 0 to 30 or less,
y is an integer from 0 to 10 or less,
l is 0 to 0.25,
m is 0.10 to 0.95,
n is 0.01 to 0.40,
o is a rational number from 0.01 to 0.10, and the sum of 1, m, n and o is 1.

In the dispersant according to an embodiment of the present invention, y is 0, and R₁₇ may be hydrogen.

In the dispersant according to an embodiment of the present invention, y is an integer of 1 to 10, and R₁₇ may be one selected from the group consisting of carboxylic acid.

The molecular weight of the dispersant may be 3,000 to 50,000 g/mol.

The dispersant may further comprise an amine-based compound.

The amine-based compound may be one or two or more selected from the group consisting of methylamine, ethylamine, propylamine, butylamine, hexylamine, heptylamine, octylamine, dioctylamine, trioctylamine, t-octylamine, aminoethanol, aminopropanol, aminobutanol, aminopentanol, aminohexanol, dodecylamine, octadecylamine, tripropylamine, N,N-dimethylbenzylamine, 2-methoxyethylamine, and olylamine.

The conductive material dispersion according to an embodiment of the present invention may comprise a dispersant, a conductive material, and a dispersion medium.

The conductive material may be one selected from the group consisting of carbon nanotubes, graphene oxide, graphene nanoplates, carbon black, and reduced graphene oxide.

The dispersion medium may be one or two or more selected from the group consisting of n-methyl-2-pinolidone, dimethylformamide, dimethyl sulfoxide, xylene, toluene, hexane, heptane, octane, cyclohexane, cyclohexanone, ethyl acetate, propyl acetate, butyl acetate, methanol, ethanol, butanol, isopropyl alcohol, glycerol, isobutanol, 2-methoxy ethanol, 2-ethoxy ethanol, 2-butoxy ethanol, 2-methoxy propanol, tetrahydrofuran, formic acid, acrylonitrile, glycol ether, and water.

The conductive material dispersion according to an embodiment of the present invention may further comprise smectite clay.

The conductive material dispersion according to an embodiment of the present invention may further comprise a cellulose-based compound.

The cellulose-based compound may be one or two or more selected from the group consisting of methylcellulose, ethylcellulose, hydroxyethylcellulose, benzylcellulose, tritylcellulose, cyanoethylcellulose, carboxymethylcellulose, carboxyethylcellulose, aminoethylcellulose, nitrocellulose, cellulose ether and carboxymethylcellulose sodium salt.

The negative electrode slurry composition for a secondary battery according to an embodiment of the present invention may comprise a dispersion, a negative electrode active material, a thickener, a binder, and a solvent.

The negative electrode active material may comprise SiOX (0≤X≤2) and a carbon-based active material.

### Advantageous Effects of Invention

An objective of the present invention is to define the structure of a dispersant that facilitates dispersing conductive materials such as carbon nanotubes, graphene, and carbon black in a medium and to provide a conductive material dispersion that induces uniform dispersion in the negative electrode slurry of a secondary battery and improves electrical connection to improve the lifespan and efficiency of secondary batteries. In addition, a conductive material pre-dispersion slurry for secondary battery electrodes with improved dispersion characteristics of the conductive material can be implemented.

### BRIEF DESCRIPTION OF DRAWINGS

FIG. 1 shows the results of H-NMR of the dispersant prepared according to Synthesis Example 1, which is an embodiment of the present invention.

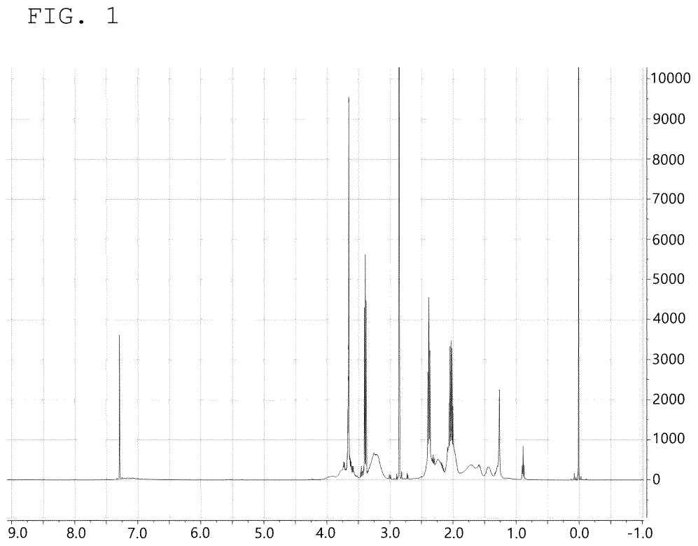

FIG. 2 shows the results of FT-IR of the dispersant prepared according to Synthesis Example 1, which is an embodiment of the present invention.

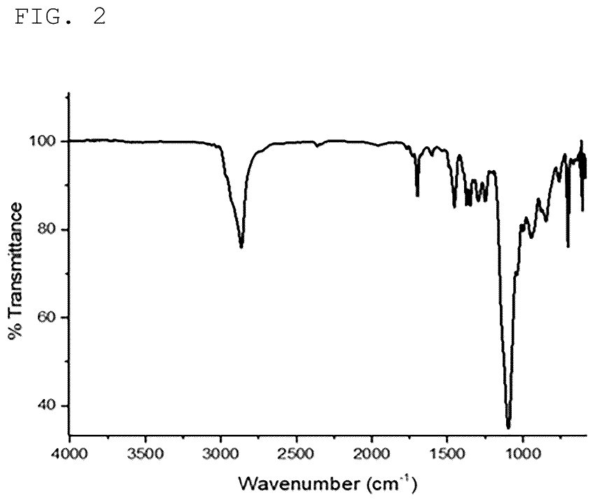

FIG. 3 shows the results of GPC of the dispersant prepared according to Synthesis Example 1, which is an embodiment of the present invention.

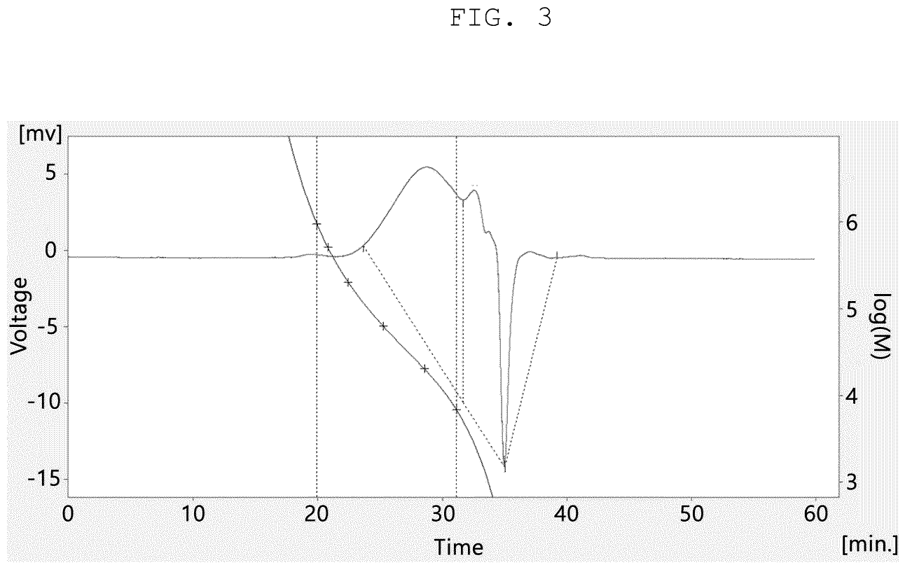

FIG. 4 shows the results of GPC of the dispersant prepared according to Synthesis Example 2, which is an embodiment of the present invention.

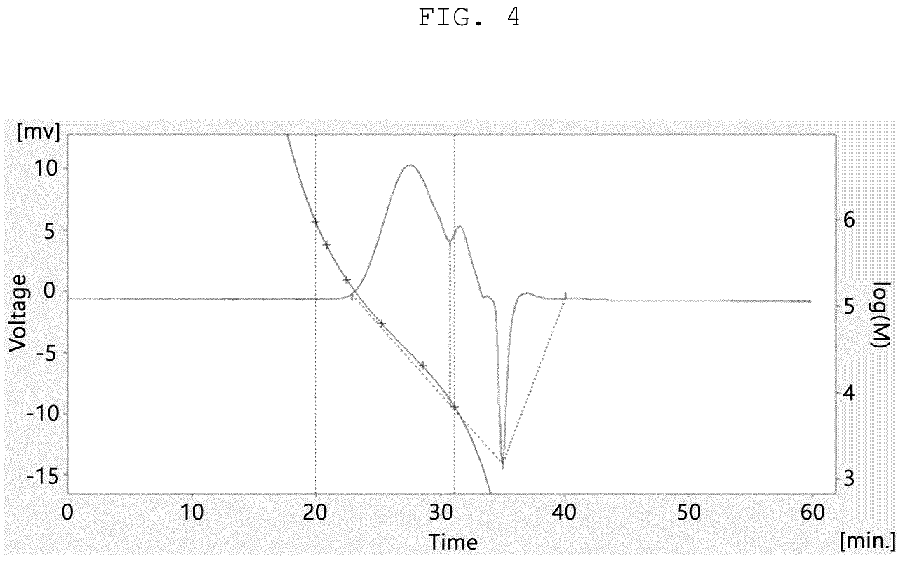

FIG. 5 shows the results of GPC of the dispersant prepared according to Synthesis Example 3, which is an embodiment of the present invention.

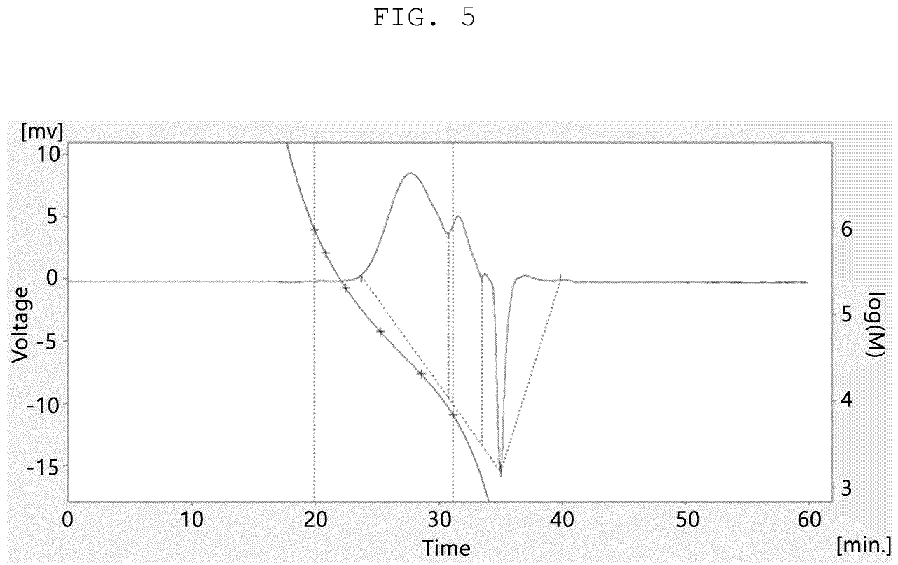

### MODE FOR INVENTION

The dispersant according to an embodiment of the present invention may be prepared by polymerizing the first to fourth monomers.

The first monomer may comprise a vinyl pinyridone-based monomer having a hydrophilic tertiary amine group. Specifically, the first monomer may be one or two or more selected from the group consisting of 1-vinyl-2-pinylidone, 5-methyl-1-vinyl-2-pinylidone, 4-methyl-1-vinyl-2-pynylidone, 3-methyl-1-vinyl-2-pinylidone, 3-chloro-1-vinyl-2-pynylidone, 4-chloro-1-vinyl-2-pynylidone, 4-chloro-1-vinyl-2-pynylidone, 3,4-dimethyl-1-vinyl-2-pinylidone, 4,5-dimethyl-1-vinyl-2-pinylidone, 3,4,5-trimethyl-1-vinyl-2-pinylidone, 4-chloro-3,5-dimethyl-1-vinyl-2-pinylidone, 3-ethyl-1-vinyl-2-pinylidone, 4-ethyl-1-vinyl-2-pinylidone, and 5-ethyl-1-vinyl-2-pynylidone.

The second monomer may comprise a hydrophilic monomer in which polyethylene glycol or polypropylene glycol is added to a vinyl or (meth) acrylate functional group. Specifically, the second monomer may be one or two or more selected from the group consisting of molyethylene glycol monoaryl ether, aryloxy (polyethylene oxide) methyl ether, aryloxy (polyethylene oxide-co-polypropylene oxide) methyl ether, polyethylene glycol mono (meth) acrylate, poly (ethylene glycol-co-propylene glycol) mono (meth) acrylate, ethoxy ethoxylate (meth) acrylate, ethoxy triglycol (meth) acrylate, polyethylene glycol monomethyl ether (meth) acrylate, and polypropylene glycol monomethyl ether (meth) acrylate.

The third monomer may comprise a vinyl monomer containing carboxylic acid. Specifically, the third monomer may be selected from the group consisting of (meth) acrylic acid and beta carboxylethyl (meth) acrylate. However, the present invention is not limited thereto, and any vinyl monomer containing carboxylic acid can be used as the third monomer.

The carboxylic acid of the third monomer may exist in a salt form, together with the vinyl pinylidone functional group of the polymer, and serve to facilitate bonding with the conductive material.

The fourth monomer may serve to lower the polarity of the dispersant by using a vinyl monomer comprising a benzene ring depending on the polarity of the medium during dispersion. In addition, the fourth monomer comprising a benzene ring may serve to increase the reliability of the secondary battery, such as withstand voltage characteristics. Specifically, the fourth monomer may be one or two or more selected from the group consisting of styrene, alpha styrene, vinyl toluene, 1-methoxy 4-vinylbenzene, 1-ethoxy 4-vinylbenzene, 1-(t-butyl)-4-vinylbenzene, 1,3,5-trimethyl-2-vinylbenzene, and 1,3,5-trichloro-2-vinylbenzene.

The dispersant according to an embodiment of the present invention may comprise the following formula 1:

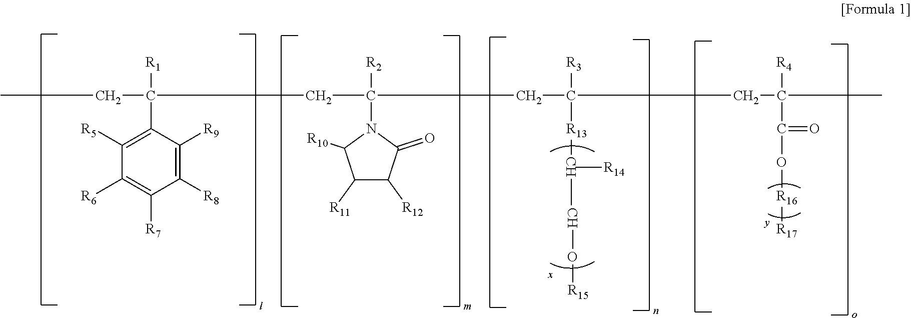

wherein R₁ to R₄ are each independently hydrogen or methane,
R₅ to R₁₂ independently comprise at least one aliphatic or aromatic hydrocarbon group or halogen element that comprises or not comprises linear, branched, or cyclic heteroatoms having 1 to 20 carbon atoms that comprise or not comprise hydrogen or unsaturated groups,
R₁₃ comprises at least one alkyl or ester group having 1 to 3 carbon atoms comprising a heterogroup,
R₁₄ is independently hydrogen or methyl,
R₁₅ is an aliphatic or aromatic hydrocarbon group that comprises or not comprises linear, branched, or cyclic heteroatoms having 1 to 20 carbon atoms that comprise or not comprise hydrogen or unsaturated groups,
R₁₆ is an aliphatic or aromatic hydrocarbon group that comprises or not comprises linear, branched, or cyclic heteroatoms having 1 to 20 carbon atoms that comprise or not comprise unsaturated groups,
R₁₇ is one selected from the group consisting of hydrogen and carboxylic acid,
x is an integer from 0 to 30 or less,
y is an integer from 0 to 10 or less,
l is 0 to 0.25,
m is 0.10 to 0.95,
n is 0.01 to 0.40,
o is a rational number from 0.01 to 0.10, and
the sum of 1, m, n and o is 1.

Specifically, the dispersant according to an embodiment of the present invention may comprise a vinyl-based dispersant defined by the following formulae 2 to 9.

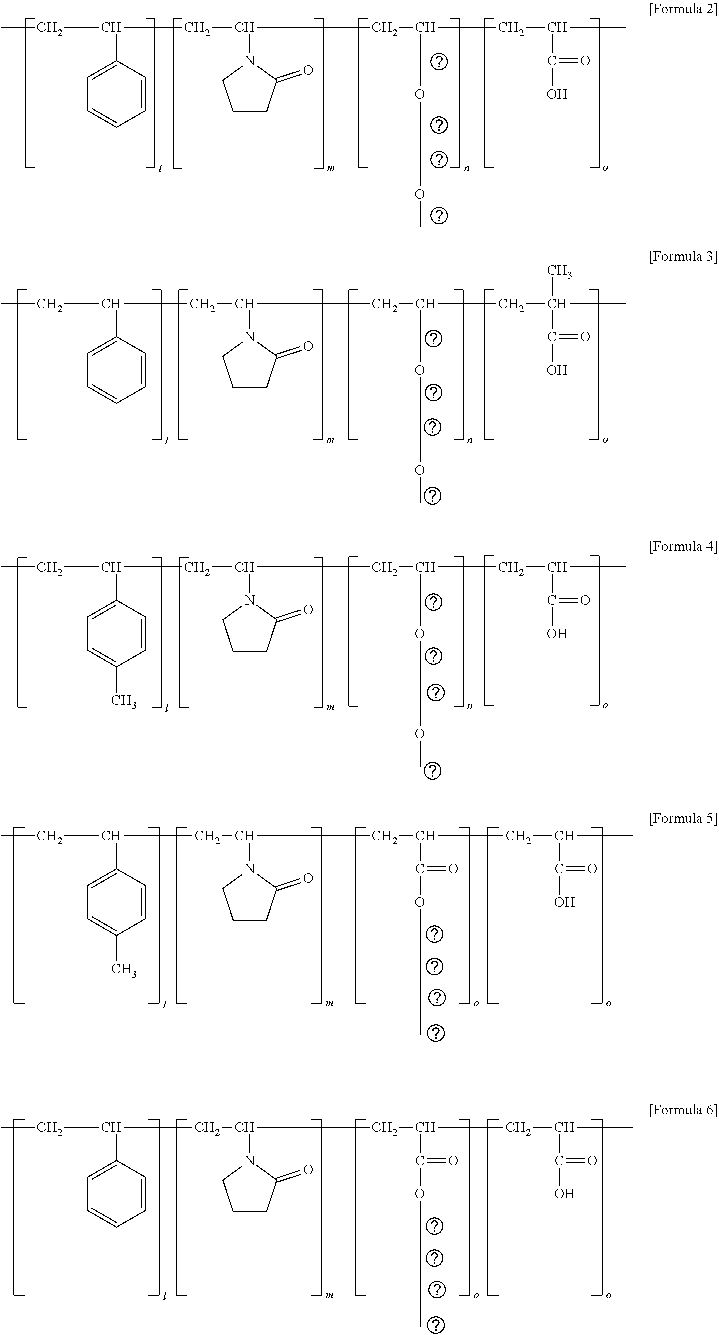

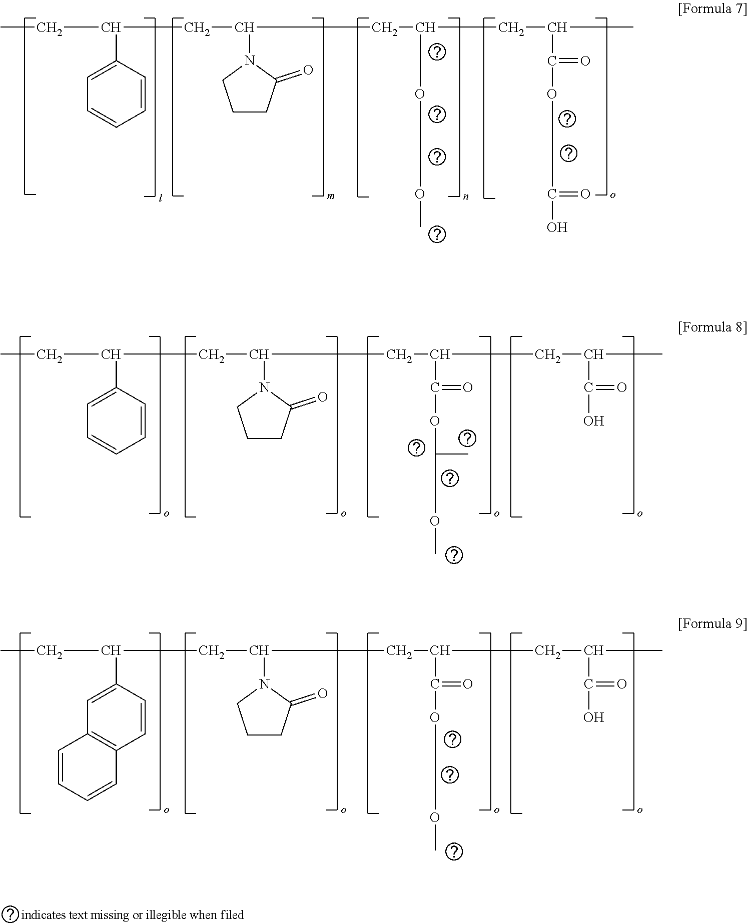

The vinyl-based polymer may be produced by appropriately selecting known production methods such as solution polymerization, bulk polymerization, emulsion polymerization, and various radical polymerizations. Preferably, it can be prepared using a solution polymerization method.

Vinyl-based copolymer resins may be produced from random copolymers, block copolymers, and graft copolymers. Preferably, it may be produced from random copolymers. This is because it is advantageous to produce same from a random copolymer in terms of cost and process.

In the solution polymerization, it is preferable to use the same solvent as the dispersion medium as a polymerization solvent. Specifically, the polymerization solvent is the same as that of the dispersion medium and may be one or two or more selected from the group consisting of n-methyl-2-pinolidone, dimethylformamide, dimethyl sulfoxide, xylene, toluene, hexane, heptane, octane, cyclohexane, cyclohexanone, ethyl acetate, propyl acetate, butyl acetate, methanol , ethanol, butanol, isopropyl alcohol, glycerol, isobutanol, 2-methoxy ethanol, 2-ethoxy ethanol, 2-butoxy ethanol, 2-methoxy propanol, tetrahydrofuran, formic acid, acrylonitrile, glycol ether and water.

In the dispersant according to an embodiment of the present invention, the molar ratio of the first monomer may be 0.1 to 0.95. Preferably, the molar ratio of the first monomer may be 0.30 to 0.9. More preferably, the molar ratio of the first monomer may be 0.4 to 0.8. If the molar ratio of the first monomer is 0.4 or less, there may be a problem that the dispersibility of the conductive material is reduced. If it is 0.8 or more, there may be a problem that mechanical properties are deteriorated in the process of manufacturing a medium and an electrode.

The molar ratio of the second monomer may be 0.01 to 0.40. Preferably, the molar ratio of the second monomer may be 0.1 to 0.35. More preferably, the molar ratio of the second monomer may be 0.15 to 0.3. If the molar ratio of the second monomer is 0.15 or less, there may be problems that compatibility with the dispersion medium decreases, and flexibility may deteriorate in the process of manufacturing an electrode. If the molar ratio of the second monomer is 0.3 or more, there may be a problem that mechanical properties are deteriorated in the process of manufacturing an electrode.

The molar ratio of the third monomer may be 0.01 to 0.10. Preferably, the molar ratio of the third monomer may be 0.02 to 0.08. If the molar ratio of the third monomer is 0.02 or less, there may be a problem that it cannot easily form a salt together with a basic compound and reduces dispersibility with the conductive material. If the molar ratio of the third monomer is 0.08 or more, there may be a problem that the dispersant resin precipitates and loses its function as a dispersant.

The molar ratio of the fourth monomer may be 0 to 0.25. Preferably, the molar ratio of the fourth monomer may be 0.10 to 0.20. If the molar ratio of the fourth monomer is 0.1 or less, there may be a problem that deterioration of mechanical properties occurs in the process of manufacturing an electrode. If the molar ratio of the fourth monomer is 0.2 or more, there may be a problem in that the dispersibility with the conductive material deteriorates.

The solution polymerization may be carried out at 12° C. to 50° C. for 5 to 30 hours under an inert gas atmosphere by adding a polymerization initiator.

The inert gas may comprise argon nitrogen, but the present invention is not limited thereto.

Polymerization initiators, chain transfer agents, emulsifiers, etc. used in the radical polymerization may be appropriately selected from widely known substances.

The dispersant according to an embodiment of the present invention may be a vinyl-based dispersant resin. The average molecular weight of the vinyl-based dispersant resin may be adjusted by the amount of polymerization initiator and chain transfer agent to be used and reaction conditions. In addition, the amount of the vinyl-based dispersant may be appropriately adjusted depending on the type of polymerization initiator and chain transfer agent.

The polymerization initiator may be one or two phases selected from the group consisting of azo-based initiators, e.g., 2,2′-azobisisobutyronitrile, 2,2′-azobis (2-amidinopropane) dihydrochloride, 2,2′-azobis [2-(5-methyl-2-imidazoline)-2-yl) propane] dihydrochloride, 2,2′-azobis (2-methylpropionamidine) disulfate, 2,2′-azobis (N,N′-dimethyleneisobutylamidine), and 2,2′-azobis [N-(2-carboxyethyl)-2-methylpropion amidine]hydrate.

The polymerization initiator may be one or two or more selected from the group consisting of peroxide-based initiators, e.g., persulfates such as potassium persulfate and ammonium persulfate, di (2-ethylhexyl) peroxydicarbonate, di (4-t-butylcyclohexyl) peroxydicarbonate, di-sec-butylperoxydicarbonate, t-butyl peroxide oxineo decanoate, t-hexylperoxypivalate, t-butylperoxypivalate, dilauroyl peroxide, di-n-octanoyl peroxide, 1,1,3,3-tetramethylbutylperoxy-2-ethylhexanoate, di (4-methylbenzoyl) peroxide, dibenzoyl peroxide, t-butylperoxyisobutyrate, 1,1-di(t-hexylperoxy) cyclohexane, and t-butyl hydroperoxide, hydrogen peroxide.

The polymerization initiator may comprise a redox-based initiator produced by combining a peroxide such as a combination of persulfate and sodium bisulfite and a combination of peroxide and sodium ascorbate, with a reducing agent. However, the present invention is not limited thereto.

The polymerization initiator may be used alone or in combination of two or more types. The amount of the polymerization initiator is preferably 1 to 15 parts by weight based on 100 parts by weight of the total amount of monomer components. More preferably, it may be 3 to 10 parts by weight.

The average molecular weight (Mw) of the vinyl-based dispersant of the resin may be adjusted depending on the amount of the polymerization initiator. As the amount of the polymerization initiator increases, the weight average molecular weight of the vinyl-based dispersant decreases. Conversely, as the amount of the polymerization initiator decreases, the weight-average molecular weight of the vinyl-based dispersant increases.

The number average molecular weight of the vinyl-based dispersant resin that is effective in the present invention may be 3,000 to 50,000. The polymerization initiator having an amount within the above-described range is preferably used to produce a dispersant having the number average molecular weight.

The vinyl-based dispersant may further comprise an amine-based compound. Dispersion stability may be maximized by using the vinyl-based dispersant and the amine-based compound simultaneously.

The carboxylic acid and amine-based compounds of the vinyl-based dispersant form a salt form to increase the dispersibility of the conductive material with a hydrophilic surface and increase the amount of the conductive material in the dispersion of the negative electrode electrolyte, thereby increasing the charging capacity of the secondary battery.

The amine-based compound may be selected from the group consisting of primary amines, secondary amines, tertiary amines, aromatic amines, and mixtures thereof, but the present invention is not limited thereto. Any types of primary amines, secondary amines, tertiary amines, and aromatic amines that are commonly used in this art may be used without any problem.

Specifically, the amine-based compound may be one or two or more selected from the group consisting of methylamine, ethylamine, propylamine, butylamine, hexylamine, heptylamine, octylamine, dioctylamine, trioctylamine, t-octylamine, aminoethanol, aminopropanol, aminobutanol, aminopentanol, aminohexanol, dodecylamine, octadecylamine, tripropylamine, N,N-dimethylbenzylamine, 2-methoxyethylamine, and oleylamine.

The amine-based compound may be mixed in advance with the vinyl-based dispersant to form a salt, or may be used together with a conductive material at a dispersing step. However, it is preferably used by mixing same with the vinyl-based dispersant in advance in order to sufficiently form a salt.

The amount of the amine-based dispersant may be 10 to 60 parts by weight, preferably 20 to 50 parts by weight, based on 100 parts by weight of the vinyl-based dispersant.

If it is below the above-described range, the dispersibility may decrease and the amount of conductive material filled may be reduced. If it is above the above-described range, the physical properties of the secondary battery may be adversely affected.

The acid value of the dispersant mixture of the amine compound and vinyl copolymer measured by a solid content-based end group analysis method may be 1 to 10 mg KOH/g, and the amine value may be in the range of 2 to 10 mg KOH/g.

The dispersant according to one embodiment of the present invention may be used alone, or may be used together with smectite clay.

Smectite is a 2:1 silicate clay mineral that has a two-dimensional space with high anisotropy, and has a large number of substitutions in the unit structure and is in the form of thin pieces to have a high surface charge and a large surface area, and has a high cation exchange capacity (CEC) and high interlayer expansion.

Smectite has the structure that Si—O tetrahedral and Al—O octahedral layers are present in a ratio of 2:1, alumina is partially substituted with the silica present in the Si—O tetrahedron, and iron or magnesium is partially substituted with alumina present in the Al—O octahedral layer. This leaves Smectite in an anion-poor state, and monovalent anions are adsorbed to the surface to balance the overall charge. At this time, if the adsorbed monovalent anions are sodium, Smectite appears as sodium montmorillonite.

Smectite-based clay is generally called bentonite, and representative examples comprise montmorillonite (MMT), nontronite, saponite, hectorite, and stevensite, the most representative being sodium montmorillonite (Na+-MMT). The smectite-based clay has the basic structure of having a combination of silica tetrahedral sheets and alumina octahedral sheets, and has various structures depending on the compositional ratio of tetrahedra and octahedrons.

Representative clays with such structure comprise Kaolin-serpentine, Pyrophyllite-Talc, mica, smectite, vermiculite, and chlorite. Among these, smectite species are further divided into montmorillonite (MMT), nontronite, saponite, and hectorite.

Cray Na+-MMT usually has the structure that one alumina octahedral sheet layer is layered between two silica tetrahedral sheets like a sandwich. The thickness of each layer is about 1 nm, and the overall size varies from several microns to 30 nm. Van der Wals forces are formed between these layers. The charge inside the layer is formed when the anions in the alumina sheet are replaced by other types of cations. For example, when Al3+ ions are substituted with Mg2+ or Fe2+, the charge in the layer forms a negative charge. Therefore, in order to balance the overall charge, exchangeable cations and water molecules are contained between the silicate layers.

MMT may increase its volume due to swelling within its crystals, and MMT, which has good swelling properties, is used in a wide variety of industries. As MMT has the unique property of swelling just by adding water, it is suitable as a host layer for interlayer cross-linked nanocomposites, and is therefore treated as an important new material with various physical properties.

It has been known that Na+-montmorillonite contains water molecules between the silicate layers, making it easy to swell with water, but other organic substances cannot penetrate between the layers.

Since clay generally has hydrophilic properties, it is organicized through appropriate surface treatment and then used to manufacture a composite with polymers. This clay organicization technology is most important for determining the physical properties of polymer nanocomposites.

In order for montmorillonite, which is commonly used to organicize clay, in an aqueous solution to control the organicization reaction of clay, it is advantageous to understand the swelling process of clay.

Montmorillonite shows regions 1, 2, and 3 depending on the concentration in aqueous solution. In region 1, the interplanar spacing increases from 1 nm to 2.2 nm while maintaining the entire crystal form in water. This is due to the hydration phenomenon of cations existing between clay layers.

On the other hand, montmorillonite which contains monovalent ions such as Li+ or Na+ shows more swelling, which is called region 2. In other words, a repulsive force is generated by the electric double layer between the clay surfaces, which is larger than the van der Waals force between the clay sheets, making it possible to separate the clay sheets. At this time, the interaction between the ends of the clay sheet and the surface is dominant, and, thus, it exhibits paste- or gel-like behavior. When the water content increases beyond region 2, the interaction between the ends of the clay sheet and the surface is eliminated, and this state is called region 3.

The attractive forces that act between the smectite clay surface organic matter comprise cationic bonding, ion-dipole bonding, dipole-dipole interaction, and π. It is confirmed that it is ion-exchanged with the hydrophilic vinyl-based dispersant used in the present invention, making dispersion with the conductive material easier and lowering the viscosity of the dispersion. The amount of smectite clay is preferably 0.01 to 0.1 parts by weight, preferably 0.02 to 0.05 parts by weight, based on 100 parts by weight of the conductive material. If the amount is below the range, the viscosity of the dispersion may be high, and if the amount is above the range, the physical properties of the secondary battery may be adversely affected.

The dispersant according to an embodiment of the present invention may be used alone or in combination with a cellulose-based compound.

The dispersibility may be improved by adding the vinyl-based dispersant and the cellulose-based compound. It is preferable to add 50 to 800 parts by weight of the cellulose compound, preferably 100 to 400 parts by weight, based on 100 parts by weight of the solid content of the vinyl-based dispersant. If it is below the range, the particle size of the conductive material may become large, and, if it is above the range, the dispersibility of the conductive material may be poor.

The weight-average molecular weight (Mw) of the cellulose-based compound may be 50,000 to 450,000 g/mol or less. Preferably, it may be 120,000 g/mol or less. More preferably, 50,000 to 120,000 g/mol of a cellulose-based compound may be used. If it is less than 50,000 g/mol, the dispersibility may be poor and the viscosity of the dispersion may increase, and, if it is more than the range, the coating properties of the slurry for electrodes may deteriorate and electrode formation may be difficult.

In addition, the degree of esterification of the cellulose-based compound used in the embodiment of the present invention may be about 0.6 to 1.0. CMC (carboxymethyl cellulose), one of the cellulose-based compounds, has three functional groups in one monomer. The degree of esterification of a cellulose-based compound indicates the extent to which the hydroxyl group among the functional groups is substituted with an ester group (i.e., degree of substitution). The degree of esterification of the cellulose-based compound may range from 0 to 3.

The degree of esterification of carboxymethyl cellulose (CMC), one of the cellulose-based compounds according to an embodiment of the present invention, may be about 0.6 to 1.0, preferably about 0.7 to 0.9. As the degree of esterification of CMC increases, the hydrophilicity of CMC may increase. If the degree of esterification is less than 0.6, the degree of hydration may be too low such that the CMC does not mix well with the polar dispersion medium, and if the degree of esterification is more than 1.0, the dispersibility with the conductive material may be reduced.

Specifically, the cellulose-based compound may be one or two or more selected from the group consisting of methylcellulose, ethylcellulose, hydroxyethylcellulose, benzylcellulose, tritylcellulose, cyanoethylcellulose, carboxymethylcellulose, carboxyethylcellulose, aminoethylcellulose, nitrocellulose, cellulose ether and carboxymethylcellulose sodium salt.

The conductive material dispersion liquid according to an embodiment of the present invention may comprise a dispersant, a conductive material, and a dispersion liquid.

The dispersant may comprise a vinyl-based dispersant prepared from the first to fourth monomers.

The conductive material may be a carbon material used as a negative electrode material for a lithium ion secondary battery. The carbon material has a low potential close to lithium and has the advantage of high charge/discharge capacity per unit mass.

The conductive material may comprise carbon black, Ketjen black, fullerene, graphene, carbon nanotubes, and carbon nanofibers. Preferably, the conductive material may be carbon nanotubes. The addition of the carbon nanotubes to a negative electrode containing graphite and silicon can achieve the reduction of electrode resistance, improvement of battery load resistance, improvement of electrode strength, and improvement of electrode expansion and contraction properties. In addition, it may improve the cycle life of lithium secondary batteries.

The average particle diameter of the carbon nanotubes may be 3 to 20 μm. The carbon nanotubes may comprise single-walled carbon nanotubes and multi-walled carbon nanotubes.

The conductive material may comprise graphene. The graphene may comprise graphene oxide, reduced graphene oxide, and graphene nanoplates.

The conductive material may comprise carbon black. The carbon black may comprise super P Conductive CarbonBlack, Ketjen Black, and Denka Black commonly used in the art.

The dispersion medium may comprise a polar solvent and a non-polar solvent. The polar solvent may comprise water. The non-polar solvent may be used in an all-solid secondary battery. Specifically, the dispersion medium may be one or two or more selected from the group consisting of n-methyl-2-pinolidone, dimethylformamide, dimethyl sulfoxide, xylene, toluene, hexane, heptane, octane, cyclohexane, cyclohexanone, ethyl acetate, propyl acetate, butyl acetate, methanol, ethanol, butanol, isopropyl alcohol, glycerol, isobutanol, 2-methoxy ethanol, 2-ethoxy ethanol, 2-butoxy ethanol, 2-methoxy propanol, tetrahydrofuran, formic acid, acrylonitrile, glycol ether and water.

A method for producing a conductive material dispersion according to an embodiment of the present invention may comprise mixing and stirring a preliminary single-walled carbon nanotube, a dispersant, and a dispersion medium. The dispersant may comprise a vinyl-based dispersant prepared from the first to fourth monomers.

It is desirable to use 1 to 30 parts by weight of the dispersant, preferably 10 to 20 parts by weight, based on 100 parts by weight of the total dispersion. If it is below the range, dispersion of the conductive material may become difficult and the conductive material may precipitate or the dispersion may thicken, and, if it is above the range, the electrical properties may deteriorate.

It is desirable to use 0.1 to 10 parts by weight of the conductive material, preferably 0.5 to 5 parts by weight, based on 100 parts by weight of the total dispersion. If it is below the range, the electrical properties are low, making it difficult to use as a negative electrode. If it is above the range, the dispersibility decreases, the viscosity increases, problems with cohesion of the conductive material occur, making it difficult to manufacture a high-quality negative electrode material.

It is desirable to use 60 to 99 parts by weight of the dispersion medium, preferably 70 to 90 parts by weight, based on 100 parts by weight of the total dispersion. If it is less than the range, the dispersibility will decrease, the viscosity will increase, agglomeration problems of the conductive material will occur, making it difficult to manufacture a high-quality positive electrode material, and, if it is more than the range, the electrical properties may be low, making it difficult to use same as a negative electrode agent.

Stirring for dispersion may be performed through at least one of mixing using a mixer and milling. The mixing may be performed using a sawblade mixer such as a homogenizer, universal stirrer, clear mixer, and fill mixer. The mixing may be performed at a rotation speed of 3,500 rpm to 4,500 rpm.

The milling may be performed by a ball mill, bead mill, basket mill, attrition mill, etc., preferably using a bead mill. When milling by the bead mill, the size of the bead mill may be appropriately determined depending on the type and amount of the conductive material and the type of dispersant. Specifically, the diameter of the bead mill may be 500 μm to 1000 μm, more specifically 500 mm to 800 μm. In addition, the bead milling process may be performed at a speed of 2,000 rpm to 4, 500 rpm and, more specifically, at a speed of 2,000 rpm to 3,000 rpm.

The negative electrode slurry according to an embodiment of the present invention may comprise the prepared dispersion, a negative electrode active material, a thickener, a binder, and a solvent.

The negative electrode active material may comprise SiOX (0≤X≤2) and a carbon-based active material. The SiOX (0≤X≤2) may be in a form containing Si and SiO₂. That is, x corresponds to the number ratio of O to Si contained in the SiOX (0≤X≤2). When SiOX (0≤X≤2) is contained, the discharge capacity of the secondary battery may be improved. The average particle diameter (D50) of SiOX may be 5 μm to 15 μm, specifically 8 μm to 12 μm. When the range is satisfied, side reactions between the SiOX and the electrolyte are suppressed, oxidation of the SiOX is controlled, and initial efficiency reduction may be prevented.

As the SiOX is contained in the negative electrode active material, the capacity of the battery may be greatly improved. However, there may be disadvantages in that SiOX has lower conductivity than graphite, etc., and its volume expands excessively when charging/discharging the battery, making it difficult to maintain a conductive path between negative active materials. To solve this problem, in one embodiment of the present invention, a specific conductive material dispersion containing single-walled carbon nanotubes is used. When using the conductive material dispersion of the present invention, the single-walled carbon nanotubes may be uniformly dispersed within the negative electrode active material layer. In addition, the single-walled carbon nanotubes are relatively thin, as compared to conventional multi-walled carbon nanotubes. Thus, despite the volume expansion of the SiOX, the electrical connection between the negative electrode active materials may be easily maintained. Accordingly, the lifespan and efficiency characteristics of the battery may be improved.

The carbon-based active material may be at least one selected from the group consisting of artificial graphite, natural graphite, and graphitized mesocarbon microbeads, and may preferably be artificial graphite.

The thickener may be one or two or more selected from the group consisting of carboxymethyl cellulose (CMC), carboxyethyl cellulose, starch, regenerated cellulose, ethyl cellulose, hydroxymethyl cellulose, hydroxyethyl cellulose, hydroxypropyl cellulose, and polyvinyl alcohol.

The binder may be one or two or more selected from the group consisting of polyvinylidene fluoride-hexafluoropropylene copolymer (PVDF-co-HFP), polyvinylidenefluoride, polyacrylonitrile, polymethylmethacrylate, polyvinyl alcohol, Carboxymethylcellulose (CMC), starch, hydroxypropylcellulose, regenerated cellulose, polyvinylpyrrolidone, tetrafluoroethylene, polyethylene, polypropylene, polyacrylic acid, ethylene-propylene-diene monomer (EPDM), sulfonated EPDM, styrene butadiene rubber (SBR), fluorine rubber, polyacrylic acid, and polymers prepared by substituting their hydrogen with Li, Na or Ca, etc., or various types of binder polymers such as various copolymers.

According to another embodiment, the present invention may comprise a conductive material dispersion for the positive and negative electrodes of a secondary battery. When it is used as a conductive material dispersion for a positive electrode, it may comprise a conductive material, a dispersant, an amine compound, and a dispersion medium. Preferably, the conductive material dispersion for the positive electrode of a secondary battery may comprise CNT as a conductive material, a vinyl-based dispersant prepared from the first to fourth monomers as a dispersant, and NMP as a dispersion medium.

When used as a conductive material dispersion for the negative electrode of a secondary battery, it may comprise a conductive material, a dispersant, and a dispersion medium. The conductive material dispersion for the negative electrode of a secondary battery may comprise a conductive material, a dispersant, a cellulose compound, and a dispersion medium. In addition, the conductive material dispersion for the negative electrode of a secondary battery may comprise a conductive material, a dispersant, smectite clay, and a dispersion medium. Specifically, it may comprise CNT as a conductive material, a vinyl-based dispersant prepared from the first to fourth monomers as a dispersant, and H₂O as a dispersion medium.

The dispersant according to an embodiment of the present invention may be prepared by polymerizing the first to fourth monomers. As a polymerization method, known production methods such as solution polymerization, bulk polymerization, emulsion polymerization, and various radical polymerizations may be appropriately selected. Preferably, the dispersant may be produced using a nuclear polymerization method.

The first monomer may comprise a vinyl pinyridone-based monomer having a hydrophilic tertiary amine group. Specifically, the first monomer may be one or two or more selected from the group consisting of 1-vinyl-2-pinylidone, 5-methyl-1-vinyl-2-pinylidone, 4-methyl-1-vinyl-2-pynylidone, 3-methyl-1-vinyl-2-pinylidone, 3-chloro-1-vinyl-2-pynylidone, 4-chloro-1-vinyl-2-pynylidone, 4-chloro-1-vinyl-2-pynylidone, 3,4-dimethyl-1-vinyl-2-pinylidone, 4,5-dimethyl-1-vinyl-2-pinylidone, 3,4,5-trimethyl-1-vinyl-2-pinylidone, 4-chloro-3,5-dimethyl-1-vinyl-2-pinylidone, 3-ethyl-1-vinyl-2-pinylidone, 4-ethyl-1-vinyl-2-pinylidone, 5-ethyl-1-vinyl-2-pynylidone.

The second monomer may comprise a hydrophilic monomer in which polyethylene glycol or polypropylene glycol is added to a vinyl or (meth) acrylate functional group. Specifically, the second monomer may be one or two or more selected from the group consisting of polyethylene glycol monoaryl ether, aryloxy (polyethylene oxide) methyl ether, aryloxy (polyethylene oxide-co-polypropylene oxide) methyl ether, polyethylene glycol mono (meth) acrylate, poly (ethylene glycol-co-propylene glycol) mono (meth) acrylate, ethoxy ethoxylate (meth) acrylate, ethoxy triglycol (meth) acrylate, polyethylene glycol monomethyl ether (meth) acrylate, polypropylene glycol monomethyl ether (meth) acrylate.

The third monomer may comprise a vinyl monomer comprising carboxylic acid. Specifically, the third monomer may be selected from (meth) acrylic acid and beta carboxylethyl (meth) acrylate. However, the present invention is not limited thereto, and any vinyl monomer comprising carboxylic acid may be used as the third monomer.

The carboxylic acid of the third monomer exists in a salt form together with the vinyl pinylidone functional group of the polymer and may serve to facilitate bonding with the conductive material.

The fourth monomer may serve to lower the polarity of the dispersant by using a vinyl monomer comprising a benzene ring depending on the polarity of the medium during dispersion. In addition, the fourth monomer comprising a benzene ring may serve to increase the reliability of the secondary battery, such as withstand voltage characteristics. Specifically, the fourth monomer may be one or two or more selected from the group consisting of styrene, alpha styrene, vinyl toluene, 1-methoxy 4-vinylbenzene, 1-ethoxy 4-vinylbenzene, 1-(t-butyl)-4-vinylbenzene, 1,3,5-trimethyl-2-vinylbenzene, and 1,3,5-trichloro-2-vinylbenzene.

The amount of the first monomer may be 0.1 to 0.95. Preferably, the molar ratio of the first monomer may be 0.30 to 0.9. More preferably, the molar ratio of the first monomer may be 0.4 to 0.8. If the molar ratio of the first monomer is 0.4 or less, the dispersibility with the conductive material is poor, and, if the molar ratio is 0.8 or more, a problem may occur in which mechanical properties are deteriorated in the process of manufacturing the medium and electrode.

The molar ratio of the second monomer may be 0.01 to 0.40. Preferably, the molar ratio of the second monomer may be 0.1 to 0.35. More preferably, the molar ratio of the second monomer may be 0.15 to 0.3. If the molar ratio of the second monomer is 0.15 or less, compatibility with the dispersion medium may decrease and flexibility may deteriorate in the process of manufacturing an electrode. If the molar ratio of the second monomer is 0.3 or more, there may be a problem that mechanical properties deteriorate in the process of manufacturing an electrode.

The molar ratio of the third monomer may be 0.01 to 0.10. Preferably, the molar ratio of the third monomer may be 0.02 to 0.08. If the molar ratio of the third monomer is 0.02 or less, there may be a problem that it is difficult to form a salt with a basic compound, resulting in reduced dispersibility with the conductive material. If the molar ratio of the third monomer is 0.08 or more, there may be a problem that the dispersant resin precipitates and loses its function as a dispersant.

The molar ratio of the fourth monomer may be 0 to 0.25. Preferably, the molar ratio of the fourth monomer may be 0.10 to 0.20. If the molar ratio of the fourth monomer is 0.1 or less, there may be a problem that mechanical properties deteriorate in the process of manufacturing an electrode. If the molar ratio of the fourth monomer is 0.2 or more, there may be a problem that the dispersibility with the conductive material is reduced.

A method for producing a conductive material slurry for the positive electrode of a secondary battery according to an embodiment of the present invention may comprise adjusting the particle size of carbon nanotubes through a ball mill process, stirring the vinyl-based dispersant, amine-based compound, dispersion medium, and carbon nanotubes at room temperature to prepare a conductive material dispersion, and processing the mixed solution at a high pressure 2 to 5 times using a diamond cell in a high pressure disperser.

The ball mill process may include a wet ball mill or a dry ball mill.

The stirring may be carried out at room temperature and 550 RPM for 2 to 4 hours.

The dispersion medium may comprise NMP.

The high pressure may be a pressure of 1,000 Mpa.

A method for producing a conductive material slurry for the negative electrode of a secondary battery according to an embodiment of the present invention may comprise adjusting the particle size of carbon nanotubes through a ball mill process, stirring a vinyl polymer, a cellulose polymer, a solvent, and the carbon nanotubes at room temperature to prepare a conductive material dispersion, and processing the mixed solution at a high pressure 2 to 5 times using a diamond cell in a high pressure disperser.

The ball mill process may include a wet ball mill or a dry ball mill.

The stirring may be carried out at room temperature and 550 RPM for 2 to 4 hours.

The dispersion medium may comprise water.

The high pressure may be a pressure of 1,500 Mpa.

### SYNTHESIS EXAMPLE 1

### Synthesis of Vinyl-Based Dispersant

100 parts by weight of n-methyl-2-pinolidone was added to a reaction vessel equipped with a gas introduction tube, thermometer, condenser, and stirrer, and it was substituted with nitrogen gas. The inside of the reaction vessel was heated to 75° C., and a mixture prepared by mixing 3.0 parts by weight of styrene, 7.0 parts by weight of acrylic acid, 20 parts by weight of 1-vinyl-2-pinylidone, 7 parts by weight of polyethylene glycol monomethyl ether (meth) acrylate (GEO Bisomer S10W) and 2.5 parts of 2,2′-azo Bis (2,4-dimethylvaleronitrile) was added dropwise over 3 hours to perform a polymerization reaction. Afterwards, it was further polymerized at 75° C. for 1 hour, and added with 0.5 part of 2,2′-azobis (2,4-dimethylvaleronitrile) again, and polymerized at 75° C. for an additional hour. The process was continued to obtain a dispersant. At this time, the number average molecular weight in terms of polystyrene measured using GPC (gel permeation chromatography) was 25,551 g/mol.

### SYNTHESIS EXAMPLE 2

### Synthesis of Vinyl-Based Dispersant

Synthesis was performed in the same manner as in Synthesis Example 1, except that 6 parts by weight of 2,2′-azobis (2,4-dimethylvaleronitrile) was used. At this time, the number average molecular weight in terms of polystyrene measured using GPC was 3.671 g/mol.

### SYNTHESIS EXAMPLE 3

### Synthesis of Vinyl-Based Dispersant

A dispersant was synthesized in the same manner as in Synthesis Example 1, except that 4.0 parts by weight of vinyl toluene, 5.0 parts by acrylic acid, 15 parts by weight of 1-vinyl-2-pinylidone, 10 parts by weight of polyethylene glycol monoaryl ether (Client, Polyglykol A 500), and 2,2′-azobis (2,4-dimethylvaleronitrile) 4 parts by weight were used. At this time, the number average molecular weight in terms of polystyrene measured using GPC was 12,268 g/mol.

### EXAMPLE 1

### Preparation of Conductive Material Slurry for Positive Electrode of Secondary Battery

The particle size of the carbon nanotubes was adjusted using a wet ball mill. The wet ball mill used 3-pie zirconia balls, and 1 g of vinyl polymer (1)+amine-based polymer dispersant was added to 94 g of NMP solvent and then dissolved. Afterwards, 5 g of carbon nanotubes were added and the mixture was rotated at 550 RPM at room temperature for 3 hours to prepare a pre-dispersion of carbon nanotubes. Zirconia balls were removed from the prepared pre-dispersion, and this was processed 2 to 5 times in a high pressure disperser at a pressure of 1,000 MPa using a 200 um diamond cell to prepare a conductive material slurry for the positive electrode of a secondary battery.

### EXAMPLE 2

### Preparation of Conductive Material Slurry for Negative Electrode of Secondary Battery

The particle size of the carbon nanotubes was adjusted using a wet ball mill. The wet ball mill used 3-pie zirconia balls, and 92.5 g of water as a solvent and 4.5 g of a vinyl polymer+cellulose polymer as a dispersant were first dissolved. Afterwards, 3 g of carbon nanotubes were added and the mixture was rotated at 550 RPM at room temperature for 3 hours to prepare a pre-dispersion of carbon nanotubes. Zirconia balls were removed from the prepared pre-dispersion, and this was processed 2 to 5 times in a high pressure disperser at a pressure of 1, 500 MPa using a 100 um diamond cell to prepare a conductive material slurry for the negative electrode of a secondary battery.

### EXAMPLE 3

### Preparation of Standard Positive Electrode

PVDF as a binder and NMP as a solvent were mixed using a rotating/revolving mixer to prepare a mixture. Afterwards, the mixture and the dispersion of the conductive material for the positive electrode of a secondary battery were mixed using a rotating/revolving mixer. Afterwards, a positive electrode active material (Umicore, NCM622) was added and mixed using a rotating/revolving mixer to prepare a positive electrode slurry. The solid content of the positive electrode slurry was 60% by weight. The weight ratio o the positive electrode active material, conductive material, and binder in the positive electrode slurry was 94:3:3.

Next, a blader was used on aluminum foil with a thickness of 20 μm, which serves as a current collector, to form an electrode plate. Next, it was dried at 120° C. for 10 hours using a vacuum oven, and rolled using a roll press to produce a standard positive electrode having a composite layer with the density of 2.8 g/cc.

### EXAMPLE 4

### Preparation of Standard Negative Electrode

A conductive material dispersion for the negative electrode of a secondary battery, carbon black, and SiOx as a silicon-based active material were mixed using a rotating/revolving mixer. Afterwards, the mixture and artificial graphite were added and mixed using a rotating/revolving mixer. Afterwards, the mixture was mixed with water as a solvent and CMC as a thickener, and styrenebutadiene as a binder was mixed using a rotating/revolving mixer to prepare a negative electrode slurry. The weight ratio of the active material (artificial graphite: SiOx=86.4:9.6), conductive material (carbon black: single-walled carbon nanotube=0.94:0.06 weight ratio), thickener, and binder in the negative electrode slurry was 96:1:1.7:1.3.

Next, a blader was used on a 10um thick copper foil that served as a current collector to form an electrode plate. Next, it was dried at 120° C. for 10 hours using a vacuum oven, and rolled using a roll press to produce a standard negative electrode having a composite layer with the density of 1.6 g/cc.

### EXPERIMENTAL EXAMPLE 1

### Measurement of Molecular Weight of Dispersant

The molecular weight of the vinyl-based dispersant prepared according to the synthesis examples was measured using gel chromatography (GPC) under the following conditions:
Device: HLC-8120GPC (Dososa)
Column: TSK-GELG4000HXL+ TSK-GELG2000HXL serial connection
Column temperature: 40° C.
Mobile phase solvent: tetrahydrofuran (THF)
Flow rate: 1.0 ml/min
Detector: R.I detector
Measured sample concentration: 0.6 wt % in THF
Standard materials for calibration: TSK STANDARD POLYSTYRENE F-40, F-4, F-1, A-2500, and A-500 (manufactured by Tosoh Corporation)

### EXPERIMENTAL EXAMPLE 2

### Evaluation on Physical Properties, Stability and Cell Test of Conductive Material Dispersion for Positive Electrode of Secondary Battery

The physical properties and stability of the conductive material dispersion prepared by polymerizing the vinyl polymer, carbon nanotubes, and solvent prepared according to Synthesis Examples 1 to 3 were evaluated.

The conductive material dispersion was prepared by varying the conditions of the content of the carbon nanotube and the type and content of the dispersant.

### 1) Measurement of Particle Size Distribution of Conductive Material Dispersion of Secondary Battery

The conductive material dispersion for a secondary battery was left in a constant temperature and humidity chamber at 25° C. for more than 30 minutes and then sufficiently stirred with 10% by weight of NMP as a positive electrode solvent and 90% by weight of H₂O as a negative electrode solvent and diluted. The cumulative particle size D50 of the conductive material dispersion for a secondary battery was measured using a particle size analyzer (Microtrack, Model S3500). It was carried out by diluting same so that the loading index value was in the range of 0.002 to 0.004.

### 2) Measurement of Viscosity of Conductive Material Dispersion of Secondary Battery

The conductive material dispersion for a secondary battery was left in a constant temperature bath at 25° C. for more than 30 minutes and stirred sufficiently, and viscosity was then measured using a viscometer (Brookfield, Model DV2T). The rotation speed RPM was adjusted to meet the conditions of 50 Torque, and the measurement was carried out using Spindle No. 4.

### 3) Evaluation on Stability of Conductive Material Dispersion of Secondary Battery

The conductive material dispersion for a secondary battery was left in a constant temperature and humidity chamber at 25° C. for 90 days and stirred sufficiently, and the rotation speed RPM was adjusted to meet the conditions of 50 Torque using a viscometer, and measurement was carried out using Spindle No. 4.

### 4) Cell Test of Conductive Material Dispersion of Secondary Battery

A 2032 coin cell type lithium-ion half cell was produced. Lithium was used as a counter electrode for each of the standard positive electrode and standard negative electrode, which were manufactured in a glove box (Korea Research Institute). The lithium ion half cell was installed in a constant temperature and humidity chamber at 25° C., and charge/discharge measurements were performed using a charge/discharge device (Wona Tech, WBCS3000). 0.1 C charge/0.1 C discharge was repeated three times and 0.5 C charge/0.5 C discharge was performed to evaluate charge/discharge characteristics.

### <Cell Test Evaluation Table>

◯ (Good): Cycle characteristics of 85% or more
Δ (Normal): Cycle characteristics of greater than 75% and less than 85%
X (defect): Cycle characteristics of less than 75%

Table 1 below shows evaluation on the particle size, viscosity, stability, and cell test of the conductive material dispersion for the positive electrode of a secondary battery.

TABLE 1

| | | | | | Particle | | | |
|---|---|---|---|---|---|---|---|---|
| | Concentration | | | | size | | | |
| | (%) of | Type of | Content of | | (D50: | Viscosity | Stability | Cell |
| | CNT | dispersant | dispersant | Solvent | um) | (cPs) | (90 days) | test |
| 1 | 5 | Vinyl-based | 1 | NMP | 10.44 | 517.4 | 650.0 | X |
| | | polymer (1) | | | | | | |
| 2 | 5 | Vinyl-based | 2 | NMP | 10.42 | 414.5 | 520.4 | X |
| | | polymer (1) | | | | | | |
| 3 | 5 | Vinyl-based | 4 | NMP | 10.27 | 330.9 | 421.4 | Δ |
| | | polymer (1) | | | | | | |
| 4 | 5 | Vinyl-based | 1 | NMP | 10.78 | 321.4 | 366.7 | ◯ |
| | | polymer (2) | | | | | | |
| 5 | 5 | Vinyl-based | 2 | NMP | 10.55 | 293.5 | 341.1 | Δ |
| | | polymer (2) | | | | | | |
| 6 | 5 | Vinyl-based | 4 | NMP | 10.51 | 255.2 | 280.5 | Δ |
| | | polymer (2) | | | | | | |
| 7 | 5 | Vinyl | 1 | NMP | 10.99 | 299.2 | 380.2 | ◯ |
| | | polymer (1) + | | | | | | |
| | | amine-based | | | | | | |
| | | polymer | | | | | | |
| 8 | 5 | Vinyl | 2 | NMP | 11.10 | 230.6 | 288.8 | Δ |
| | | polymer (1) + | | | | | | |
| | | amine-based | | | | | | |
| | | polymer | | | | | | |
| 9 | 5 | Vinyl | 4 | NMP | 10.94 | 193.3 | 231.7 | Δ |
| | | polymer (1) + | | | | | | |
| | | amine-based | | | | | | |
| | | polymer | | | | | | |
| 10 | 5 | Vinyl | 1 | NMP | 10.21 | 141.0 | 170.5 | ◯ |
| | | polymer (2) + | | | | | | |
| | | amine-based | | | | | | |
| | | polymer | | | | | | |
| 11 | 5 | Vinyl | 2 | NMP | 10.19 | 108.2 | 121.5 | ◯ |
| | | polymer (2) + | | | | | | |
| | | amine-based | | | | | | |
| | | polymer | | | | | | |
| 12 | 5 | Vinyl | 4 | NMP | 10.03 | 88.5 | 102.0 | Δ |
| | | polymer (2) + | | | | | | |
| | | amine-based | | | | | | |
| | | polymer | | | | | | |
| 13 | 10 | Vinyl-based | 1 | NMP | 10.42 | 603.3 | 702.0 | X |
| | | polymer (1) | | | | | | |
| 14 | 10 | Vinyl-based | 2 | NMP | 10.42 | 510.3 | 622.7 | Δ |
| | | polymer (1) | | | | | | |
| 15 | 10 | Vinyl-based | 4 | NMP | 10.40 | 444.5 | 540.0 | Δ |
| | | polymer (1) | | | | | | |
| 16 | 10 | Vinyl-based | 1 | NMP | 10.77 | 410.1 | 493.6 | ◯ |
| | | polymer (2) | | | | | | |
| 17 | 10 | Vinyl-based | 2 | NMP | 10.52 | 366.6 | 421.6 | ◯ |
| | | polymer (2) | | | | | | |
| 18 | 10 | Vinyl-based | 4 | NMP | 10.55 | 320.1 | 394.8 | Δ |
| | | polymer (2) | | | | | | |
| 19 | 10 | Vinyl | 1 | NMP | 10.98 | 345.5 | 420.2 | ◯ |
| | | polymer (1) + | | | | | | |
| | | amine-based | | | | | | |
| | | polymer | | | | | | |
| 20 | 10 | Vinyl | 2 | NMP | 10.66 | 297.0 | 341.9 | ◯ |
| | | polymer (1) + | | | | | | |
| | | amine-based | | | | | | |
| | | polymer | | | | | | |
| 21 | 10 | Vinyl | 4 | NMP | 10.76 | 210.4 | 255.1 | Δ |
| | | polymer (1) + | | | | | | |
| | | amine-based | | | | | | |
| | | polymer | | | | | | |
| 22 | 10 | Vinyl | 1 | NMP | 10.10 | 224.6 | 310.2 | ◯ |
| | | polymer (2) + | | | | | | |
| | | amine-based | | | | | | |
| | | polymer | | | | | | |

### EXPERIMENTAL EXAMPLE 3

### Evaluation on Physical Properties, Stability, and Cell Test of Conductive Material Dispersion for Negative Electrode of Secondary Battery

The vinyl polymer prepared according to Synthesis Examples 1 to 3, carbon nanotubes, and a solvent were polymerized to prepare a conductive material dispersion and evaluate its physical properties and stability. The evaluation method was carried out in the same manner as in Experimental Example 2 above.

The conductive material dispersion solution was prepared by varying the conditions of the content of carbon nanotubes and the type and content of the dispersant.

Table 2 below shows evaluation on the particle size, viscosity, stability, and cell test of the conductive material dispersion for the negative electrode of a secondary battery.

TABLE 2

| | | | | | Particle | | | |
|---|---|---|---|---|---|---|---|---|
| | Concentration | | | | size | | | |
| | (%) of | Type of | Content of | | (D50: | Viscosity | Stability | Cell |
| | CNT | dispersant | dispersant | Solvent | um) | (cPs) | (90 days) | test |
| 1 | 1 | Vinyl-based | 0.5 | H₂O | 11.43 | 984.2 | 1112.4 | Δ |
| | | polymer | | | | | | |
| 2 | 1 | Vinyl-based | 1.5 | H₂O | 11.52 | 826.8 | 925.7 | Δ |
| | | polymer | | | | | | |
| 3 | 1 | Vinyl-based | 2.0 | H₂O | 11.42 | 680.1 | 766.3 | Δ |
| | | polymer | | | | | | |
| 4 | 1 | Cellulose | 0.5 | H₂O | 11.64 | 360.0 | 384.0 | ◯ |
| | | polymer | | | | | | |
| 5 | 1 | Cellulose | 1.5 | H₂O | 11.42 | 410.4 | 442.1 | ◯ |
| | | polymer | | | | | | |
| 6 | 1 | Cellulose | 2.0 | H₂O | 11.40 | 522.3 | 551.0 | Δ |
| | | polymer | | | | | | |
| 7 | 1 | Smectite | 0.1 | H₂O | 10.99 | 71.9 | 94.5 | ◯ |
| | | clay | | | | | | |
| 8 | 1 | Smectite | 0.2 | H₂O | 10.93 | 25.2 | 31.1 | ◯ |
| | | clay | | | | | | |
| 9 | 1 | Smectite | 0.3 | H₂O | 10.88 | <10 | <10 | ◯ |
| | | clay | | | | | | |
| 10 | 3 | Vinyl-based | 0.5 | H₂O | 11.86 | 1,320.4 | 1,662.1 | ◯ |
| | | polymer | | | | | | |
| 11 | 3 | Vinyl-based | 1.5 | H₂O | 11.77 | 1,023.2 | 1,352 | ◯ |
| | | polymer | | | | | | |
| 12 | 3 | Vinyl-based | 2.0 | H₂O | 11.67 | 911.4 | 1,190.4 | Δ |
| | | polymer | | | | | | |
| 13 | 3 | Cellulose | 0.5 | H₂O | 10.33 | 422.0 | 430.0 | ◯ |
| | | polymer | | | | | | |
| 14 | 3 | Cellulose | 1.5 | H₂O | 10.22 | 623.5 | 634.1 | ◯ |
| | | polymer | | | | | | |
| 15 | 3 | Cellulose | 2.0 | H₂O | 10.34 | 796.3 | 810.2 | Δ |
| | | polymer | | | | | | |
| 16 | 3 | Smectite | 0.1 | H₂O | 11.55 | 241.6 | 290.1 | ◯ |
| | | clay | | | | | | |
| 17 | 3 | Smectite | 0.2 | H₂O | 11.32 | 198.0 | 241.1 | ◯ |
| | | clay | | | | | | |
| 18 | 3 | Smectite | 0.3 | H₂O | 11.60 | 144.2 | 170.0 | Δ |
| | | clay | | | | | | |
| 19 | 1 | Vinyl | 1.5 + 1.0 | H₂O | 11.44 | 289.9 | 361.4 | Δ |
| | | polymer + | | | | | | |
| | | cellulose | | | | | | |
| | | polymer | | | | | | |
| 20 | 1 | Vinyl | 1.5 + 2.0 | H₂O | 11.32 | 355.2 | 421.9 | Δ |
| | | polymer + | | | | | | |
| | | cellulose | | | | | | |
| | | polymer | | | | | | |
| 21 | 1 | Vinyl | 1.5 + 3.0 | H₂O | 11.34 | 405.2 | 460.0 | Δ |
| | | polymer + | | | | | | |
| | | cellulose | | | | | | |
| | | polymer | | | | | | |
| 22 | 1 | Vinyl | 1.5+ 0.1 | H₂O | 10.76 | 133.3 | 185.4 | ◯ |
| | | polymer + | | | | | | |
| | | Smectite | | | | | | |
| | | clay | | | | | | |
| 23 | 1 | Vinyl | 1.5 + 0.2 | H₂O | 10.20 | 104.4 | 130.6 | ◯ |
| | | polymer + | | | | | | |
| | | Smectite | | | | | | |
| | | clay | | | | | | |
| 24 | 1 | Vinyl | 1.5 + 0.3 | H₂O | 10.19 | 88.4 | 102.0 | Δ |
| | | polymer + | | | | | | |
| | | Smectite | | | | | | |
| | | clay | | | | | | |
| 25 | 3 | Vinyl | 1.5 + 1.0 | H₂O | 10.60 | 673.6 | 747.2 | ◯ |
| | | polymer + | | | | | | |
| | | cellulose | | | | | | |
| | | polymer | | | | | | |
| 26 | 3 | Vinyl | 1.5 + 2.0 | H₂O | 10.23 | 722.3 | 903.5 | ◯ |
| | | polymer + | | | | | | |
| | | cellulose | | | | | | |
| | | polymer | | | | | | |
| 27 | 3 | Vinyl | 1.5 + 3.0 | H₂O | 10.22 | 810.0 | 1020.1 | ◯ |
| | | polymer + | | | | | | |
| | | cellulose | | | | | | |
| | | polymer | | | | | | |
| 28 | 3 | Vinyl | 1.5 + 0.1 | H₂O | 10.31 | 203.6 | 254.4 | ◯ |
| | | polymer + | | | | | | |
| | | Smectite | | | | | | |
| | | clay | | | | | | |
| 29 | 3 | Vinyl | 1.5 + 0.2 | H₂O | 10.34 | 178.0 | 191.0 | ◯ |
| | | polymer + | | | | | | |
| | | Smectite | | | | | | |
| | | clay | | | | | | |
| 30 | 3 | Vinyl | 1.5 + 0.3 | H₂O | 10.33 | 130.2 | 152.7 | Δ |
| | | polymer + | | | | | | |
| | | Smectite | | | | | | |
| | | clay | | | | | | |

1. A dispersant comprising the following formula 1: 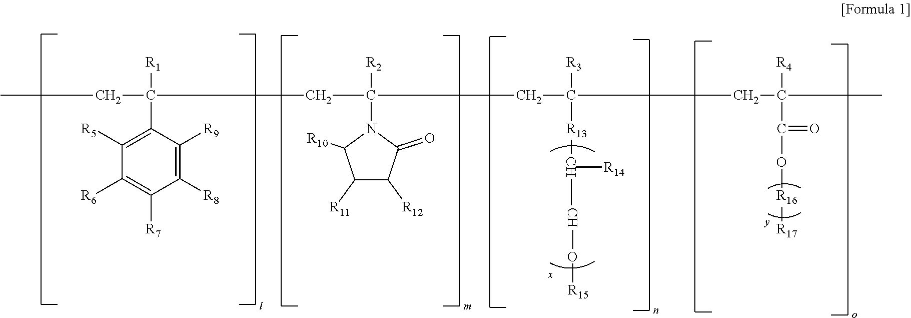
wherein R₁ to R₄ are each independently hydrogen or methane,
R₅ to R₁₂ independently comprise at least one aliphatic or aromatic hydrocarbon group or halogen element that comprises or not comprises linear, branched, or cyclic heteroatoms having 1 to 20 carbon atoms that comprise or not comprise hydrogen or unsaturated groups,
R₁₃ comprises at least one alkyl or ester group having 1 to 3 carbon atoms comprising a heterogroup,
R₁₄ is independently hydrogen or methyl,
R₁₅ is an aliphatic or aromatic hydrocarbon group that comprises or not comprises linear, branched, or cyclic heteroatoms having 1 to 20 carbon atoms that comprise or not comprise hydrogen or unsaturated groups,
R₁₆ is an aliphatic or aromatic hydrocarbon group that comprises or not comprises linear, branched, or cyclic heteroatoms having 1 to 20 carbon atoms that comprise or not comprise unsaturated groups,
R₁₇ is one selected from the group consisting of hydrogen and carboxylic acid,
x is an integer from 0 to 30 or less,
y is an integer from 0 to 10 or less,
l is 0 to 0.25,
m is 0.10 to 0.95,
n is 0.01 to 0.40,
o is a rational number from 0.01 to 0.10, and
the sum of 1, m, n and o is 1.

2. The dispersant according to claim 1, wherein y is 0, and R₁₇ is hydrogen.

3. The dispersant according to claim 1, wherein y is an integer from 1 to 10, and R₁₇ is one selected from the group consisting of carboxylic acid.

4. The dispersant according to claim 1, wherein the number molecular weight of the dispersant is 3,000 to 50,000 g/mol.

5. The dispersant according to claim 1, further comprising an amine-based compound.

6. The dispersant according to claim 5, wherein the amine-based compound is one or two or more selected from the group consisting of methylamine, ethylamine, propylamine, butylamine, hexylamine, heptylamine, octylamine, dioctylamine, trioctylamine, t-octylamine, aminoethanol, aminopropanol, aminobutanol, aminopentanol, aminohexanol, dodecylamine, octadecylamine, tripropylamine, N,N-dimethylbenzylamine, 2-methoxyethylamine, and olylamine.

7. A conductive material dispersion comprising a dispersant according to claim 1, a conductive material, and a dispersion medium.

8. The conductive material dispersion according to claim 7, wherein the conductive material is one selected from the group consisting of carbon nanotubes, graphene oxide, graphene nanoplates, carbon black, and reduced graphene oxide.

9. The conductive material dispersion according to claim 7, wherein the dispersion medium is one or two or more selected from the group consisting of n-methyl-2-pinolidone, dimethylformamide, dimethyl sulfoxide, xylene, toluene, hexane, heptane, octane, cyclohexane, cyclohexanone, ethyl acetate, propyl acetate, butyl acetate, methanol, ethanol, butanol, isopropyl alcohol, glycerol, isobutanol, 2-methoxy ethanol, 2-ethoxy ethanol, 2-butoxy ethanol, 2-methoxy propanol, tetrahydrofuran, formic acid, acrylonitrile, glycol ether, and water.

10. The conductive material dispersion according to claim 7, further comprising smectite clay.

11. The conductive material dispersion according to claim 7, further comprising a cellulose-based compound.

12. The conductive material dispersion according to claim 11, wherein the cellulose-based compound is one or two or more selected from the group consisting of methylcellulose, ethylcellulose, hydroxyethylcellulose, benzylcellulose, tritylcellulose, cyanoethylcellulose, carboxymethylcellulose, carboxyethylcellulose, aminoethylcellulose, nitrocellulose, cellulose ether and carboxymethylcellulose sodium salt.

13. A negative electrode slurry composition for a secondary battery comprising a dispersion according to claim 7, a negative electrode active material, a thickener, a binder, and a solvent.

14. The negative electrode slurry composition for a secondary battery according to claim 13, wherein the negative electrode active material comprises SiOX (0≤X≤2) and a carbon-based active material.
